(19) United States
(12) Patent Application Publication
(10) Pub. No.: US 2022/0313767 A1
(43) Pub. Date: Oct. 6, 2022

(54) Herbal composition for metabolic syndromes and method of treatment thereof

(71) Applicant: Christopher Kitalong, Koror (PW)

(72) Inventor: Christopher Kitalong, Koror (PW)

(21) Appl. No.: 17/739,241

(22) Filed: May 9, 2022

Related U.S. Application Data
(62) Division of application No. 16/258,599, filed on Jan. 27, 2019.

(51) Int. Cl. A61K 36/83; A61K 31/7048; A61K 31/155; A61K 31/64; A61P 3/10

(52) U.S. Cl. CPC A61K 36/83; A61K 31/7048; A61K 31/155; A61K 31/64; A61P 3/10; A61K 2236/331

(57) ABSTRACT

The present invention relates to an herbal composition for the treatment of metabolic syndromes in a subject in need thereof, said composition comprising Phaleria nisidai; optionally along with hypoglycemic agents, a process thereof, and a method for the treatment of metabolic syndromes.

This patent application claims the benefit of priority, under 35 U.S.C. Section 121, to U.S. Non-Provisional patent application Ser. No. 16/258,599, filed Jan. 27, 2019, entitled “HERBAL COMPOSITION FOR METABOLIC SYNDROMES AND METHOD OF TREATMENT THEREOF” which are incorporated by reference herein in their entirety.

### FIELD OF THE PRESENT INVENTION

The present invention relates to an herbal composition for the treatment of metabolic syndromes, and also a process of preparing the herbal composition.

### BACKGROUND AND PRIOR ART REFERENCES

Metabolic syndrome is a clustering of at least three of the five following medical conditions: central obesity, high blood pressure, high blood sugar, high serum triglycerides, and low serum high-density lipoprotein (HDL). Metabolic syndrome is associated with the risk of developing cardiovascular disease and type 2 diabetes (Kaur J, 2014). In the US about a quarter of the adult population has metabolic syndrome, and the prevalence increases with age, with racial and ethnic minorities being particularly affected (Beltran-Sanchez et al., 2013).

Insulin resistance, metabolic syndrome, and pre-diabetes are closely related to one another and have overlapping aspects. Diabetes Mellitus is the most common endocrine disease. This disease is characterized by poor regulation of blood glucose levels in human beings. Blood glucose is the source of energy for basic cell functions. This glucose is driven to the cell by insulin, which is secreted by the pancreas. Diabetes Mellitus is caused by inadequate insulin secretion by the pancreas or the resistance generated by insulin receptors to the insulin. Therefore, this disease is characterized by a metabolic abnormality. Diabetes is a major metabolic disorder in which the body does not produce or properly use insulin and is characterized by hyperglycemia, glycosuria, hyperlipidemia, negative nitrogen balance and sometimes ketonemia. Diabetes is one of the most common diseases affecting human population today.

The Republic of Palau, a relatively isolated island in the Pacific, has a very fast rising rate of non-communicable diseases (particularly obesity, diabetes mellitus type II and hypertension), making it a well-suited place for research on this theme. Local high-level commitment is observed in the country, since the President of Palau declared a state of emergency on non-communicable diseases in 2011. Many public health interventions at both individual and population levels have been implemented in order to overcome the problem of obesity and lifestyle-related diseases. These programs have had very limited success and innovative approaches need to be explored (Ichiho et al., 2013).

In a country-wide population study, Delal A Kar, a Palauan traditional drink made with Phaleria nisidai and other plants, was recorded as the local treatment with the best reported outcome in case of diabetes in Palau, hence it could be selected for further clinical study (Graz et al., 2015).

Prior phytochemical analysis of Phaleria nisidai Kaneh (or P. nisidae) has revealed the presence of flavones, benzophenones (Kitalong et al., 2007, 2012), xanthones (Matsuda 2005, Kitalong et al., 2007, 2012), acylglucosylsterols (Matsuda et al., 2005) fatty acids (Kitalong et al., 2007) and daphnane diterpene esters (Kulakowski et al., 2015). There is a high mangiferin (FIG. 1) content in P. nisidai aqueous extract (Kitalong et al. 2012). From Hou's study (Hou et al., 2012) on mangiferin and sitagliptin in diabetic rats, several interesting properties of mangiferin were observed: improved glucose tolerance test, inhibition of DPPIV enzyme, increased insulin secretion and increased β-cell/islet area ratio. In addition, the following properties of mangiferin were observed in animal studies: alpha amylase inhibitory effect (IC50 value 74.35±1.9 μg/ml) and alpha glucosidase inhibitory effect (IC50 41.88±3.9 μg/ml) when compared with standard drug acarbose (IC50 83.33±1.2 μg/ml) This is immediate reaction, reduce glucose uptake in the gut so compound should be present at all points when food is in gut. Therefore, 0-3 hrs (digestive process). (Dineskumar et al., 2010). Further, unpublished β-cell studies on mangiferin showed increased insulin production between 15-30 min. period and increasingly reduced insulin at 1 hr. Major issues with the use of pure mangiferin is solubility, which has been an issue in most biological models (Acosta et. al 2016).

As shown in our previous RTO study, Palauans commonly use the local recipe of interest and volunteers were recruited and agreed to join the randomized clinical study. This provided an opportunity to measure clinical effects of P. nisidai using standard clinical study methods (Graz et al., 2007). One problem in designing such a comparative study of a local medicine well known in a small population is developing a credible placebo. Thus, the study objective was not only to develop a protocol and finding preliminary results but was also to properly test the placebo quality. With these objectives, DAK was assessed for the first time as adjuvant therapy for patients with insufficient diabetes control) Furthermore, prior to developing the clinical trial initial mechanistic approaches were pursued within a small sample group. Unpublished data shows that there was a flattening of the glucose spike, per OGTT, in both normal and diabetic/pre-diabetic patients. Furthermore, insulin and DDPIV levels were measured in aforementioned patients, and showed fast acting insulin secretion and later DDP4 inhibition. This information was presented to the Palau IRB for approval for clinical trial. ¹According to the American Diabetes Association, “lowering A1C to below or around 7% has been shown to reduce microvascular and neuropathic complications of type 1 and type 2 diabetes. Therefore, for microvascular disease prevention, the A1C goal for nonpregnant adults in general is <7%. (grade A) (Ref.: Standards of Medical Care in Diabetes—2009. Diabetes Care January 2009 32:S6S12)

### Objects of the Present Invention

The main object of the present invention is to develop an herbal composition for the treatment of metabolic syndromes.

Another main object of the present invention is to develop a process for the preparation of the herbal composition from Phaleria nisidai for the treatment of metabolic syndromes.

Yet another object of the present invention is to develop a method of treating metabolic syndromes, using the herbal composition from Phaleria nisidai.

Still another object of the present invention is to develop a method of treating metabolic syndromes, using the herbal composition for adjuvant therapy with hypoglycemic agents.

### SUMMARY OF THE PRESENT INVENTION

The present invention relates to an herbal composition for the treatment of metabolic syndromes in a subject in need thereof, said composition-comprising Phaleria nisidai, optionally along with hypoglycemic agents, a process thereof and also, a method of metabolic syndromes.

### BRIEF DESCRIPTION OF THE DRAWINGS

FIG. 1 represents structure of the mangiferin.

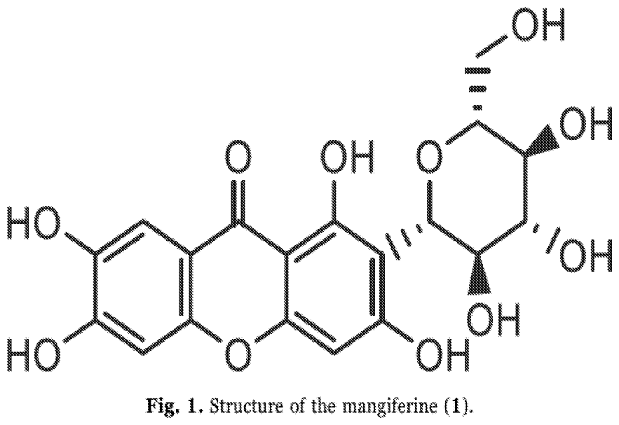

FIG. 2 represents study flow: enrolment of the participants and completion of the clinical trial.

FIG. 3 represents UHPLC-UV-ELSD-TOF-MS analysis of the water decoction of leaves of Phaleria nisidai. A) ELSD, B) UV at 254 nm and C) HRMS-TOF in negative ionization.

FIG. 4a represent time to insulin quantities; active component showing immediate stimulation higher than positive control and then lowering of stimulation after 60 min.

FIG. 4b Represents glucose-mediated beta-cell stimulation with higher insulin quantities at lower doses (65 μg/ml) over baseline (white column) and positive control (black column).

FIG. 5 represents quantification of mangiferin by different extraction methods, Calibration curve of mangiferin (R2=0.99788), and chromatographic profiles of representative P. nisidai extracts [(A) methanol, (B) aqueous, and (C) traditional].

FIG. 6 represents quantification after extraction clinical trial leaf samples; the number in the beginning of the label indicates whether it was the first, second or third extraction of the same leaves (meaning extracted then fluid removed, new fluid added and extracted again, etc.).

FIG. 7 represents baseline data of patients included in final analysis.

FIG. 8 represents min clinical results at the beginning, after 6 weeks and 12 weeks of study (means).

FIG. 9 represents mean of symptom score for every patient and in both groups.

FIG. 10 represent after two years of follow up with 27 of the original 55 patients, who followed the same treatment regime, the average drop in HbA1C was 2.49 and median was 2.34.

### DETAILED DESCRIPTION OF THE PRESENT INVENTION

Accordingly, the present invention relates to an herbal composition for the treatment of metabolic syndromes in a subject in need thereof, said composition-comprising Phaleria nisidai; optionally along with hypoglycemic agents, a process thereof and also, a method of metabolic syndromes.

In still another embodiment of the present invention, wherein the herbal composition is in extraction form and any derivative of this form.

In still another embodiment of the present invention, wherein the herbal composition is in decoction form and any derivative of this form.

In still another embodiment of the present invention, wherein the herbal composition is used for adjuvant therapy with hypoglycemic agents.

In still another embodiment of the present invention, wherein the hypoglycemic agents are Metformin, Glyburide, Glucovanse, or Micronase.

In still another embodiment of the present invention, wherein a method for preparation of the herbal composition obtained from plant Phaleria nisidai; wherein the composition is prepared by a standardized extraction method.

In still another embodiment of the present invention, wherein the standardized extraction method is decoction.

In still another embodiment of the present invention, wherein the composition is prepared by the ratio of the plant Phaleria nisidai and water 1˜10:10˜2,000 (w/v), ideally 6:1,000 (w/v).

In still another embodiment of the present invention, wherein the composition is prepared by boiling in water, the boiling time is 1 min. to 3 hours, ideally 30 min. to 1 hour.

In still another embodiment of the present invention, wherein a method of treatment for a subject for the treatment of metabolic syndromes, comprising a step of administrating to said subject a composition comprising hypoglycemic agent as a first active ingredient and the herbal composition as claimed in claim 1 as an adjuvant active ingredient.

In still another embodiment of the present invention, wherein the said subject is administered the composition before meal from 1 min. to 3 hours, ideally 3 min. to 1 hr.

In still another embodiment of the present invention, wherein the said subject is administered the composition before meal from 1 min. to 2 hours.

In still another embodiment of the present invention, wherein the said subject is administered the composition before meal from 1 min. to 1 hour.

The instant invention is not only novel but also, inventive in nature. The comparative results of Phaleria nisidai powder (whole), and the composition of the instant application are shown below to establish inventiveness of the application as well as the mode of administering; pre-meal to increase effectiveness due to the fast-acting and quickly metabolized nature of the active compound shown below.

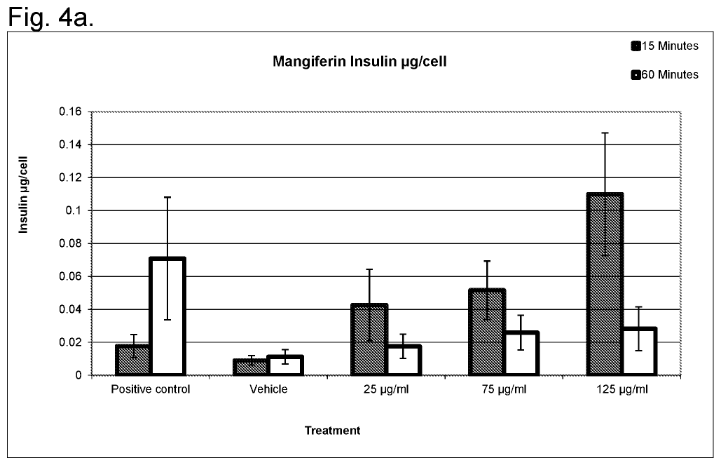

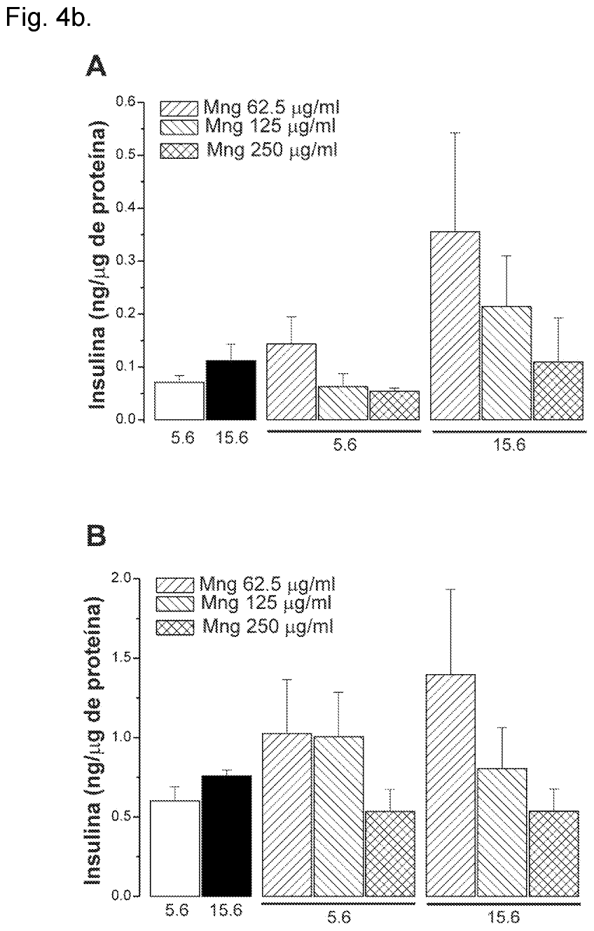

FIG. 4a shows time to insulin quantities; active component showing immediate stimulation higher than positive control and then lowering of stimulation after 60 min. Furthermore, FIG. 4b shows a lower dosing of active component showing peak insulin levels in a glucose-mediated manner over positive control.

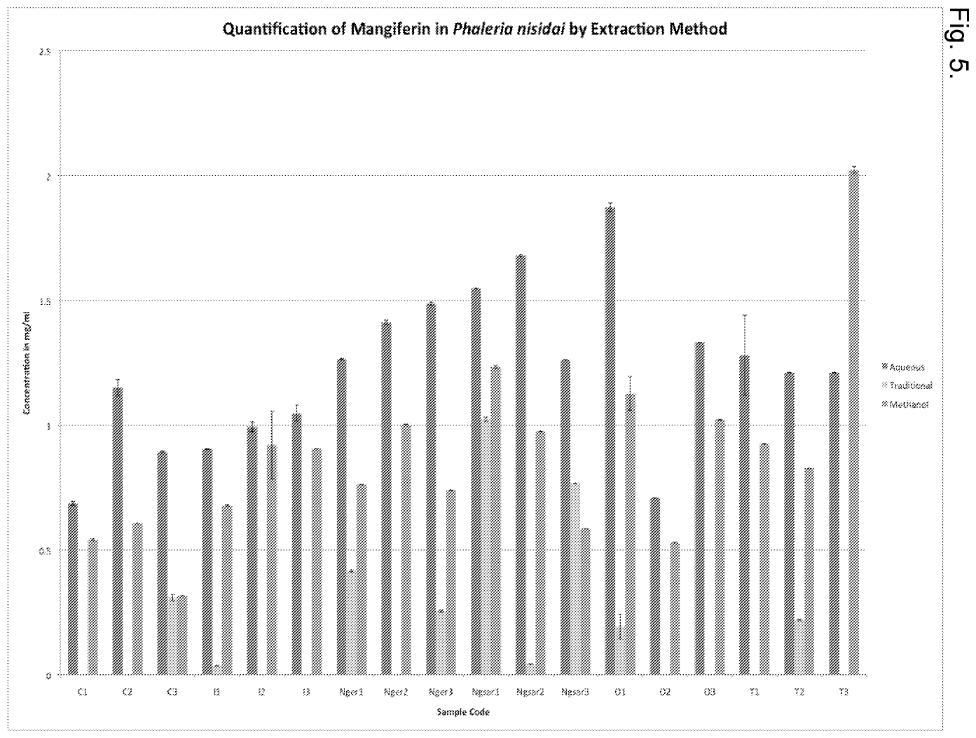

FIG. 5 shows that the overall quantity of marker/active compound is more efficiently extracted in water than organic solutions, furthermore aqueous extractions can be moved more easily to therapy. This shows ideal extraction of mangiferin through water for therapy as well as quantities in whole/organic solvent extractions.

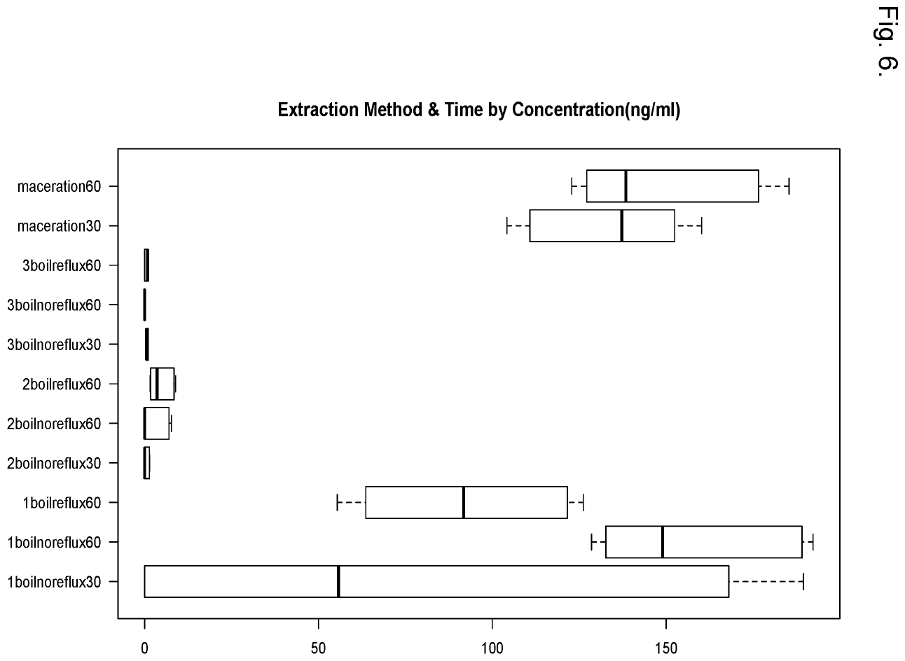

FIG. 6 shows quantification after extraction 1, 2 and 3 of clinical trial leaf samples; quantity drops drastically after initial extraction of marker compound from all samples.

Maceration shows that the leaves were simple crushed and left in water to extract; as seen the marker compound and tea quantity drops drastically after the first extraction; maceration which can be considered time 0 show proper extraction. This shows that extraction time can vary from 0/1 min meaning heat extraction to 60 min heat extraction. But the ideal extraction was boiling with no reflux 1 time for 30-60 min.

The aforementioned figures clearly show that the inventors have been able to reach the right mode of administering of the Phaleria nisidai, as well as proper boiling time and repetitions. In addition, the right mode of administering is further supported by the inventiveness in using appropriate concentration ranges of the composition, as well as the biological activity time window for therapy. This makes the invention both novel and inventive in nature.

The aforementioned results clearly establish synergy of the composition. The activity is much more that the mere additive effect of individual components. Rather, it is very clearly reflected in the data that the non-active components are bringing down the activity of the active components. Therefore, elimination of the inactive components led to significant increase in the overall activity. Thus, the composition is not only effective in new cases of diabetes mellitus, but also, would be effective in management of patients that are not responsive to metformin and sufonyl-ureas.

Therefore, the patentability requirements of novelty, inventiveness, and utility are satisfied. The current method of management of blood sugar in diabetes mellitus lends itself for long-term use due to the lack of side effects. Long-term complications of Diabetes are also well known and lifestyle changes are necessary for maintaining controlled blood sugar to reduce and prevent progression of diabetes mellitus type 2. Due to relative responsiveness to treatments, there is always a need for development of new therapies for the management of blood sugar. This therapy offers novel, holistic, methodology backed by scientific evidence.

Phaleria nisidai is known to possess anticholesterol and antidiabetic activities. However the compounds responsible for effective antidiabetic properties have not been clearly elucidated and studied in detail in human subjects. Phaleria nisidai contains, among several compounds such as: benzophone O and C glycosides, xanthone O and C glycosides, flavones and flavonoid glycosides as well as other polar and semi-polar molecules.

Apparently, some of these in combination are responsible for antidiabetic properties and some others in combination are responsible for lipid lowing qualities.

Oral hypoglycemic drugs are used only in the treatment of type 2 diabetes which is a disorder involving resistance to secreted insulin. Type 1 diabetes involves a lack of insulin and requires insulin for treatment. There are now four classes of hypoglycemic drugs: Sulfonylureas; Metformin; Thiazolidinediones; Alpha-glucosidase inhibitors. These drugs are approved for use only in patients with type 2 diabetes and are used in patients who have not responded to diet, weight reduction, and exercise. They are not approved for the treatment of women who are pregnant with diabetes. The present invention is about identification of individual compounds of Phaleria nisidai that have beneficiary effect in the case of type 2 diabetes. The present invention illustrates the method for extraction of select compounds from Phaleria nisidai resulting in a combination of certain molecules that act in tandem; and with synergy; and leads to effective control of blood glucose in subjects suffering from type 2 diabetes. The present invention explains the composition of matter of the extract derived from Phaleria nisidai leaves.

### Experiment 1: Phaleria Nisidai Kaneh Leaves Collection

The leaves were collected throughout the study duration in Palau. Reference specimens are held at Belau National Museum Herbarium and New York Botanical Garden herbarium. Non-UV dried leaves were used for easier storage, reproducibility and standardization of product.

### Experiment 2: Preparation of DAK

The tested product, DAK, is smallest common denominator of several Delal A Kar recipes: a standardized decoction of Phaleria nisidai Kaneh., or Ongael in Palauan, leaves prepared following a traditional recipe. 60 grams of dry P. nisidai leaves for 10 liters of water were brewed in an induction boiler for one hour and allowed to cool down to room temperature. Once at room temperature the decoction was filtered and transferred in 500 mL new plastic bottles and sealed. All processes were completed at the PAIR brewing facility, using leaves from the same trees dried in non-UV conditions.

It shows use of plant at 5-7 leaves per gallon—as the pot we had was 10 L maximum we calculated the average weight of leaves at approximately 3.25 g, therefore we went to the higher end of the spectrum at 7 leaves: therefore 60 g/10 L was closest to 7 leaves×3.52 grams/gallon @ 2.64 gal=10 L so 7 leaves×3.52 grams×2.64 gal=60 g per 10 L. (Dahmer et al, 2012).

### Experiment 3: Mangiferin Detection

The standardized decoction was prepared according to the previously mentioned protocol for DAK and was then lyophilized.

UHPLC-UV-PDA-ELSD. UHPLC measurements were performed using an Acquity UPLC system (Waters®, Milford, Mass., USA), with a binary pumping system, an auto-sampler, a column manager with a pre-column heater, a UV-PDA and an evaporative light scattering detector (ELSD), Sedex 85 (Sedere® LT-ELSD, Alfortville, France). The system was controlled using Empower® 3 Software. UV-PDA detection was performed from 210 to 500 nm (1.2 nm resolution). The temperatures in the auto sampler and in the column oven were fixed at 10 and 40° C., respectively. The binary system was using two mobile phases: water with 0.1% formic acid (A) and acetonitrile with 0.1% formic acid (B) (ULC/MS grade, Biosolve Chimie SARL, Dieuze, France). A solution containing rutin (Fluke AG, Buchs, Switzerland) and glycyrrhetinic acid (Carl Roth, Karlsruhe, Germany) at 500 μg/mL) standards was injected before the analyses to verify measured retention times and to allow for comparison with other chromatographic devices. Separation was achieved on an Acquity UPLC BEH C₁₈ column (1.7 μm, 2.1×150 mm; Waters®, Milford, Mass., USA) with a 30 min linear gradient of 95% of A and 5% of B, followed by a 10 min isocratic step with 95% of B and a 10 min re-equilibration step. Injection volume was set at 2 μL, the flow rate was fixed at 0.46 mL/min. The lyophilized DAK was suspended in water and methanol (70/30 v/v) at 10 mg/mL.

UHPLC-UV-HRMS-TOF analysis. The metabolite profiling of the lyophilized DAK was performed on a Waters® Acquity UPLC system coupled to a Waters® Micromass LCT Premier Time-Of-Flight (TOF) mass spectrometer (Waters®), equipped with an electrospray interface (ESI). The chromatographic conditions were similar to those used for the UHPLC-UV-PDA-ELSD metabolite profiling. Analyses were performed in negative ionisation mode in the 100-1300 Da range with acquisition times of 0.3 s in centroid mode. The ESI conditions were set as followed: capillary voltage 2400 V, cone voltage 40 V, source temperature 120° C., desolvation temperature 300° C., cone gas flow 20 L/h, desolvation gas flow 800 L/h and MCP (microchannel plate) detector voltage 2450 V MassLynx software 4.1, SCN 639 (Waters®, Milford, USA) was used to drive the system. A solution containing both rutin (20 μg/mL) and glycyrrhetinic acid (10 μg/mL) was injected before the analyses to check the reliability of the measured retention time. The lyophilized DAK was resuspended in water and methanol (70/30 v/v) at 1 mg/mL.

### Experiment 4: Preparation of DAK and Placebo Plastic Bottles

The placebo drink was prepared with distilled water and food color. McCormick® Egg yellow food color with small additions of red and blue food color drops were used to create the DAK color profile in the placebo. Both DAK and placebo were double blinded, had identical appearance and kept refrigerated.

### Experiment 5: Study Protocol

The present study was a randomized, double-blind, crossover clinical trial performed in Koror, Palau at Belau Medical Clinic and PAIR office. The research protocol was based on Cochrane Collaboration recommendations for patient safety and confidentiality, following Helsinki declaration and WHO recommendations of good practices (WHO, 2004), and the study report was prepared in accordance with the CONSORT statement. The medical ethics committee of the Republic of Palau approved the protocol and each patient gave an informed consent.

Each patient included in the study: was diabetic with an HbA1C>7% or fasting plasma glucose>126 mg/dl; stable for at least for 8 weeks on prescribed conventional treatments before the beginning of study. Exclusion criteria for the study were: pregnant or lactating mothers; insulin-dependent diabetes or end-stage renal disease; co-morbid disease² such as heart disease; history ² . . . and relatively rare situations, requiring special care/attention, such as: —body mass index (BMI)>45.0 kg/m2. of comas or seizures; and liver or kidney disease. A total of 68 patients with type II diabetes were selected among 79 patients who answered to a public announcement and voluntarily enrolled in the study. —marked polydipsia and polyuria with >10% weight loss <3 months before screening, —history of diabetic ketoacidosis or hyperosmolar nonketotic coma, —cardiovascular event within 3 months of screening, —congestive heart failure New York Heart Association class III/IV, —known ejection fraction ≤40%, —history of hemoglobinopathies. Inspired by a recent RCT of diabetic treatment: White et al. BMC Endocrine Disorders 2014, 14:17.

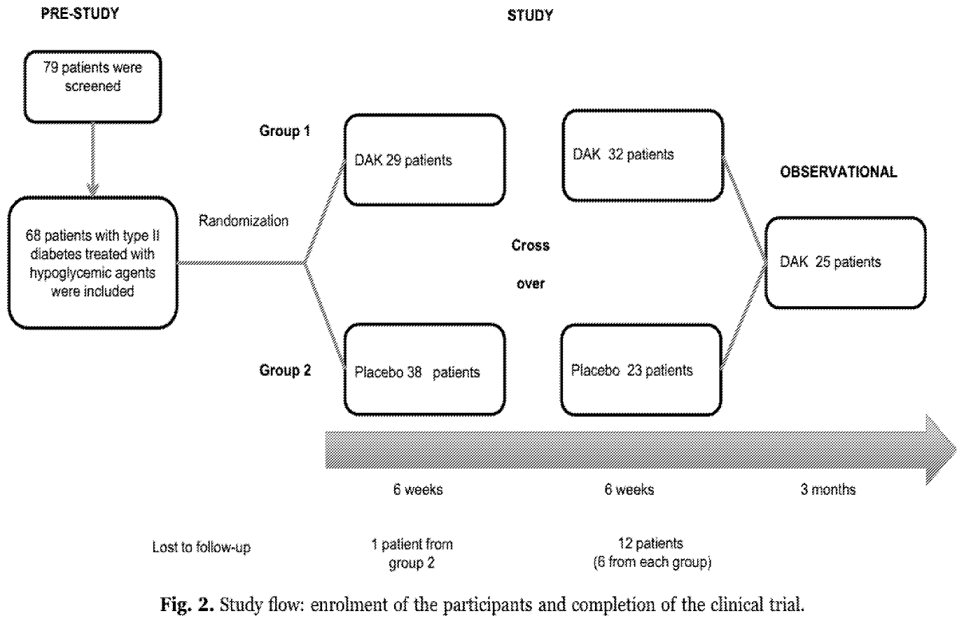

Patients were assigned group numbers from a pre-set computer-generated random table (equal size study groups). The general scheme of the study (FIG. 2) was a RCT ([usual treatment+placebo] versus [usual treatment+DAK]), with crossover after 6 weeks, without washout time between the two periods. The rationale for not having a washout period was because DAK is active in the glucose metabolism within hours (from pre-tests post-prandial glucose profile on 12 patients with and without DAK—unpublished) and the primary outcome is that HbA1C reaches a stable level, or very close, within 6 weeks of a given regimen.

Patients were examined at times 0 (baseline), 6 weeks (cross-over) and 12 weeks (end of the study). In addition, those who had accepted a longer study were followed-up drinking DAK tea for an additional 3 months period (observational).

A patient visit was performed at baseline, 6 weeks and 12 weeks to: to fill a questionnaire with personal information such as age, gender, food, activity, habits and medication; to check patients' general health; to documented noted side effects; how patients were taking the treatment; if they changed their diabetes standard treatments, etc.; as well as for trained nurses to get clinical measurements such as height, weight, waist circumference and blood pressure.

Baseline lab analysis included: triglycerides, cholesterol, uric acid, glucose, creatine, HDL-C, ALT/GPT, AST/GOT, LDL measurements (Chemray 120, Pioway Medical Lab); HbA1C (HbA1c Analyzer, Medical Source Co. Ltd), ESR (Dispette 2 saline), and urine analysis (Rayto—RT—150 Urine analyzer).

Patients were provided blinded bottles of DAK or placebo on a weekly basis by visiting nurses at the clinic, with ready-to-use product and instructions on dose: 30 mL three times a day before meals. Each participant was also provided with Nutrition 101 classes providing basic information on diet, given a checklist for meal audits and provided with and trained to use a home glucometer to easily follow their blood sugar rate.

### Experiment 6: Sample Size

This was the first clinical trial in the region, therefore, per our IRB agreement, we decided that quarter of the standard sample size would be used to determine feasibillity. The final sample size was calculated at 220, phase 3 of IRB agreement, adjusted depending on variance observed during phase 2. Calculation were done in Stata software: and to be able to detect a difference of 15% (considered clinically significant) of the main outcome measures (e.g. 10% & 25% change in HbA1c), with alpha set at 0.05 (two-sided) and power 0.8 (20 patients lost to follow-up), we wanted to include at least 55 patients, plus 10% to adjust for drop-out rate.

### Experiment 7: Outcomes

Primary outcome of measures of diabetes control are that HbA1C changes from baseline to 6 weeks and from 6 to 12 weeks for every patient. HbA1C, glycated haemoglobin, was chosen as a common indicator of diabetes control, directly proportional to average blood glucose concentration in the past few weeks (Østoft et al., 2014). We also compared HbA1C changes from baseline to 12 weeks.

Secondary outcome measures are the proportion of patients achieving normalized blood sugar after the 12 week study (HbA1C<7.0% and/or HbA1C<6.5%). We also assessed changes from baseline to 12 weeks on: fasting plasma glucose; body weight and waist circumference in addition to measures, at times 0, 6 and 12 weeks, of blood pressure; triglycerids; total cholesterol; HDL and LDL.

Treatment adherence and blinding success were also assessed and reported. A symptom score was built with followings propositions: “being tired/feeling a loss of energy”, “gaining weight”, “losing weight”, “being thirsty more than usual”, “waking up to urinate at night”, “suffering from frequent infections”, “headache”, “weak/lack of strength”, or “urge to eat even when not hungry”. General health, quality of life and lifestyle changes were assessed as part of another study and will be published elsewhere.

### Experiment 8: Statistical Analyses

Statistical analyses were conducted under the “intention to treat” model to estimate effectiveness of DAK. First, A paired t-test was used to assess a change between week 12 and baseline. DAK effect was estimated by computing the differences between results after DAK preparation and baseline: achieved by computing result at week 6 and baseline for those patients in DAK/placebo group and by the difference between week 12 and baseline for those in placebo/DAK group. Then the DAK effect was compared to the change after placebo preparation (estimated in the same way). A paired t-test of continuous variables was used to assess if the DAK (versus placebo) effect is null. Finally, the DAK effect was compared to placebo effect using a paired t-test. A Non-parametric test was used in case the distribution was non-normal and Mc Nemar test was used for discrete/nominal variables. The statistical model did not include the factor ‘sequence’, because there were too few repeated measures; nor was an adjustment for baseline differences in potential confounding factors (e.g. BMI) performed in view of their fair spread over groups at baseline.

Results

1. Participants

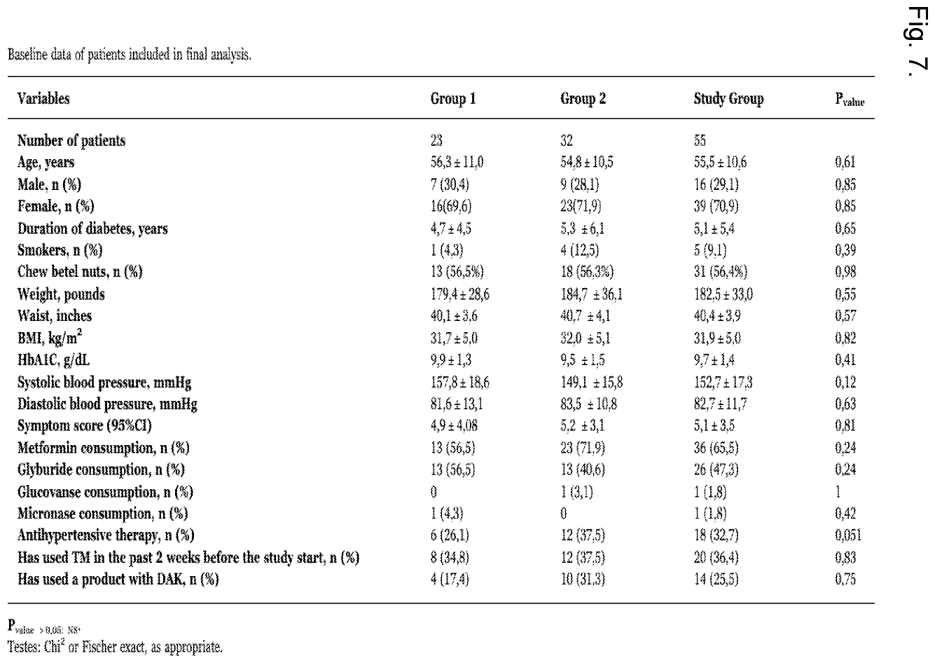

A total of 68 patients met all the inclusion criteria and 55 patients completed the study; 13 patients ceased to participate in the study during the follow-up periods (see FIG. 2). Baseline characteristics were similar and differences between groups were non-significant (FIG. 7).

2. Treatment Adherence and Placebo Quality

Patients declared through 6 weeks and 12 weeks surveys that a mean of 88% of the prescribed doses were taken (median 95%), similar in both groups (means 88 and 89%), with 2 extremes in both groups (down to between 40 and 60%)—(p=0.8). All patients came, once a week, to get their new bottle at the clinic. Usual diabetes standard treatments were continued with no change. No patient started a new diabetes treatment during the study. One patient only received antihistamines for non-related purposes.

Less than 10% correctly identified if they received placebo or DAK, which means a fairly non-recognizable placebo. Moreover, both placebo and DAK were well tolerated (no hypoglycemia, no adverse effect), while some patients (4 quotes) noticed that DAK made them hungry, however, their weight decreased.

3. Outcomes

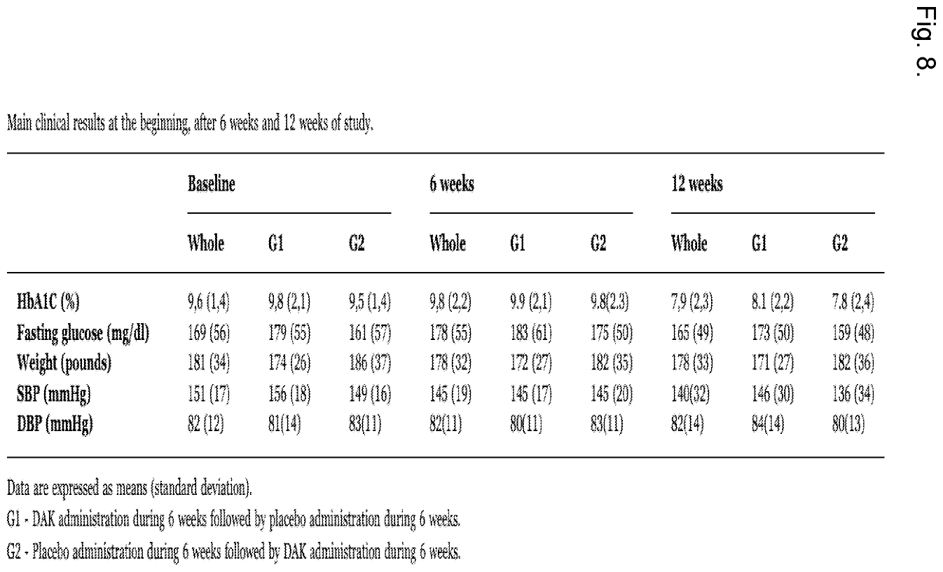

Primary outcome measure of diabetes control was that HbA1C changes from baseline (FIG. 8). There was a clinically significant change of HbA1C after 12 weeks: a mean decrease of 1.9% (SD 2.6, P<0.001). Out of 55 patients, 46 had a decrease of HbA1C after the study period. At 12 weeks, of the 55 participants 18 (33%) achieved an HbA1c<7.0%; 11 (34%) who had received DAK during the first 6-week period; and 7 (30%) who received DAK during the second 6-week period (McNemar for the whole crossover: P=0.8). After 12 weeks, out of the 55 participants 15 (27%) achieved HbA1c ≤6.5: 34% of those who had received DAK during the first 6-week period and 17% who received it during the second 6-week period (Mc Nemar: p=0.4). At 12 weeks, a total of 76% (42/55) of patients reduced their HbA1C of at least 0.5% at and 75% (41/55) at least 0.7%.

At 6 weeks, waist circumference decreased by 1 inch in both groups and at 12 weeks there was no change in those who had placebo during the first 6-week period and a 1 inch reduction in those who had DAK during the first 6-week period (p=0.1). The weight loss 95% Ci was −2 to −4.5 pounds. Triglycerides remained the same among those who had DAK for the first 6-week period (155-158-155 mg/dl at 0, 6 and 12 weeks) and tended to increase (from a mean 143 to 168 mg/dl) among those who had placebo during first 6-week period (p=0.7). Total cholesterol at baseline was 171 mg/dl (CI95% 162-179) in both groups. We did not notice any changes on total cholesterol at 6 weeks. At 12 weeks, mean cholesterol was 153 (CI95% 144-163), the same in both groups. There was no significant difference observed in HDL and LDL.

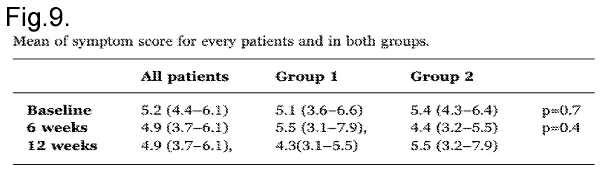

When asked “In general, how do you find your health?”, most patients evaluated their health as average at baseline (mean and median 3 in both groups on a 5 point scale, 75% and 79% gave this 3 rating in both groups). There was a slight trend toward improvement with a mean 2.9 in both groups at 6 weeks, and at 12 weeks 2.7 for group 1 (placebo during this second period) and 2.6 for group 2 (DAK between 6 and 12 weeks). The symptom score showed no statistically significant difference. (FIG. 9)

Several patients added comments on their subjective health. At 6 weeks, patients noticed that after DAK period: “body feels lighter than before”, “feeling good about myself”, “found improved memory function”, “before used to have pain inside, but now no more”, “makes me eat smaller portions.”, “it curves my appetite”, “not dizzy, less pain on my sole, bit strong”. After placebo period: “feel healthy with good appetite”, “eating habit changed, small portions and awareness of eating more vegetables”, “not always hungry”, “my body feels light and good”, “feel good”. At 12 weeks: after DAK period: “I eat less” and after placebo period: “eat more”.

One of the survey questions was about possible secondary effects due to DAK. At 6 weeks and after DAK period some patients mentioned effects “possibly caused by the treatment”: feel hungry/with urge to eat (3 patients), “head aches” (3), “blurred vision”, “sweets more”, “tired”, “tight muscles, can't walk far” (1 patient each)—all rated as mild. After placebo period, patients mentioned effects “possibly caused by the treatment”: “do not always go to the bathroom as usual in morning”, “sometimes feels cold”, “if he/she forgets to drink the treatment, doesn't feel good”—rated as mild. At 12 weeks and after the DAK period, rated as mild: a patient said she was “hardly using restroom” and the treatment made her “bloated”. Another declared “My bottle made me hungry all the time”. After the placebo period frequent urge to eat was also described by three patients and one felt he/she was “gaining weight” (rated as moderate).

Research staff (PAIR team) also made some observations. They noticed that patients were happy to have a place for health information and receive classes on diet. PAIR appeared more accessible than hospital or clinic, because it is not official and patients do not need to go through the whole process of a formal consultation. PAIR team noticed that patients were also satisfied with the glucometer they received. For physical activity, patients regretted not receiving advice from a specialist trainer. The PAIR team only suggested doing more physical activity, even if it is only walking. Finally, the confidence in Yano's clinic (which delivered bottles for patients) is such that most patients were convinced the “placebo” was an effective treatment. (as well, have to know whether, even if they knew it was a placebo, they thought it was a treatment too or that the placebo was valid).

4. Additional Observations

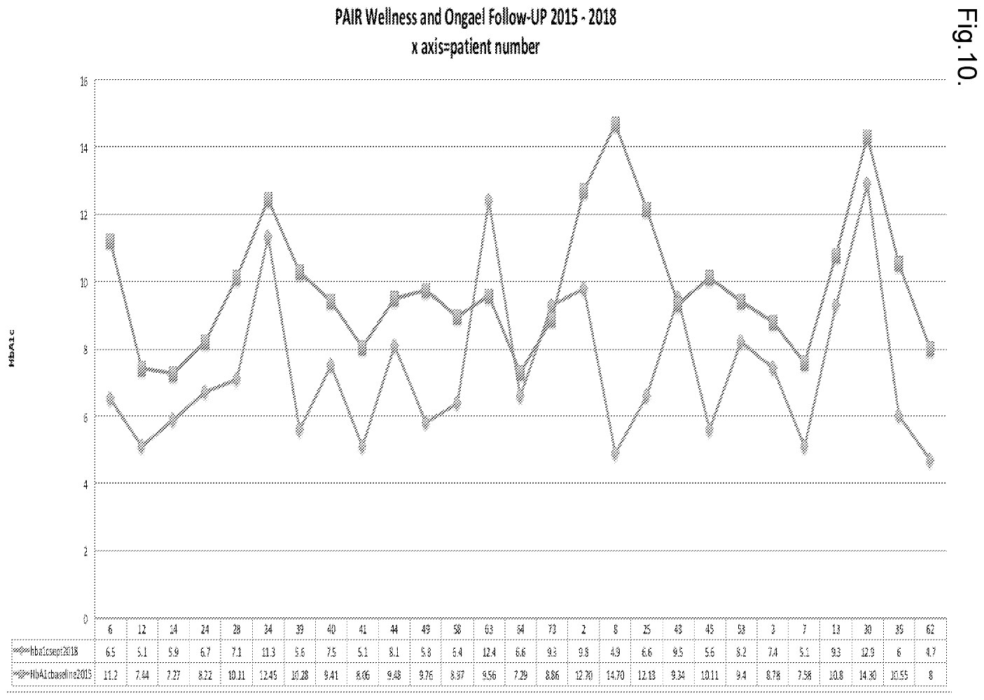

In a subsequent 3-month follow-up on 25 of the participating patients, the dosage of DAK was doubled and still well tolerated. Half of these patients maintained or improved their HbA1C reduction, a quarter had a new increase but still under baseline, 4 were back to baseline and 2 higher. After two years of follow up with 27 of the original 55 patients, who followed the same treatment regime, the average drop in HbA1C was 2.49 and median was 2.34. (FIG. 10).

5. Mangiferin Detection in DAK Preparations

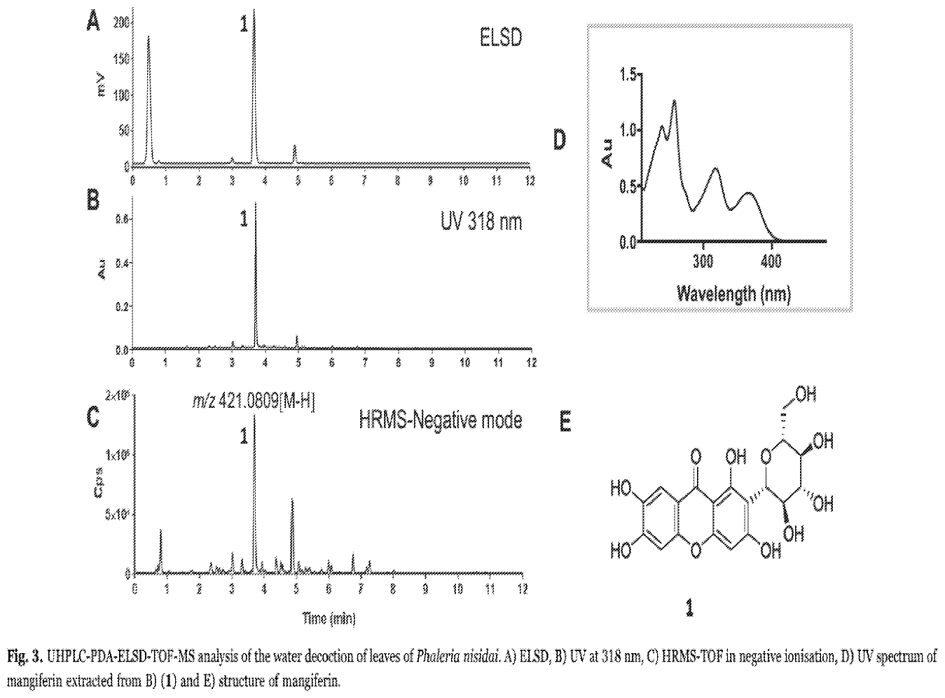

In order to verify the presence of mangiferin (1) in DAK preparation, UHPLC profiling was preformed directly on the lyophilized decoction. Detection was achieved by UV-PDA and ELSD and in separately by high-resolution mass spectrometry (ESI-HRMS-TOF). The characteristic UV-PDA and HRMS spectra recorded for the main peak eluting at 3.7 minute confirmed the presence of mangiferin (see FIG. 3). Spiking experiments were performed for validation (supplementary material). ELSD provided a semi-quantitative evaluation of the level chemical constituents in the DAK preparation. As expected from literature, mangiferin (1) was the main secondary metabolite present. The signals at the beginning of the ELSD chromatogram reflected the important content of polar compounds, mainly sugars, which are usually found in aqueous plant extracts. The PDA spectra retrieved from the UV chromatogram confirmed the presence mangiferin and of other minor xanthones.

### REFERENCES

Beltran-Sanchez H, Harhay M O, Harhay M M, McElligott S, 2013. “Prevalence and trends of metabolic syndrome in the adult U.S. population, 1999-2010”. Journal of the American College of Cardiology. 62
Dahmer, S. M., Kitalong, A. H., Balick, M. J., Kitalong, C., Herrera, K., Lee, R., Law, W., Rehuher, F., Tadao, V.-R., Hanser, S., 2012. Palau primary health care manual: combining conventional treatments and traditional uses of plants for health and healing, Palau. The New York Botanical Garden, Belau National Museum, Ministry of Health of the Republic of Palau.
Dineshkumar, B., Mitra, A., Manjunatha, A., 2010. Studies on the anti-diabetic and hypolipidemic potentials of mangiferin (xanthone glucoside) in streptozotocin-induced type 1 and type 2 diabetic model rats. Int. J. Adv. Pharm. Sci. 1, 75-85.
EI-Sayed, M. I., 2011. Effects of Portulaca oleracea L. seeds in treatment of type-2 diabetes mellitus patients as adjunctive and alternative therapy. J. Ethnopharmacol. 137, 643-651.
Graz, B., Elisabetsky, E., Falquet, J., 2007. Beyond the myth of expensive clinical study: assessment of traditional medicines. J. Ethnopharmacol. 113, 382-386.
Graz, B., Kitalong, C., Yano, V., 2015. Traditional local medicines in the republic of Palau and non-communicable diseases (NCD), signs of effectiveness. J. Ethnopharmacol. 161, 233-237.
Hosseini, S., Jamshidi, L., Mehrzadi, S., Mohammad, K., Najmizadeh, A. R., Alimoradi, H., Huseini, H. F., 2014. Effects of Juglans regia L. leaf extract on hyperglycemia and lipid profiles in type two diabetic patients: a randomized double-blind, placebo-controlled clinical trial. J. Ethnopharmacol. 152, 451-456.
Hou, J., Zheng, D., Fan, K., Yu, B., Xiao, W., Ma, J., Jin, W., Tan, Y., Wu, J., 2012. Combination of mangiferin and dipeptidyl peptidase-4 inhibitor sitagliptin improves impaired glucose tolerance in streptozotocin-diabetic rats. Pharmacology 90, 177-182.
Ichiho, H. M., Demei, Y., Kuartei, S., Aitaoto, N., 2013. An assessment of non-communicable diseases, diabetes, and related risk factors in the Republic of Palau: a systems perspective. Hawaii J. Med. Public Health 72, 98-105.
Kaur J, 2014. “A comprehensive review on metabolic syndrome”. Cardiology Research and Practice. 2014: 1-21.
Kitalong, C., Chao-mei, M., El-Halawany, A., Hattori, M., 2007. HCV Protease Inhibitory effects of the leaves of Phaleria nisidai, a Palauan folk medicine. In: Proceedings of the Japan Society of Pharmacognosy Annual Meeting. Japan Society of Pharmacognosy Annual Meeting Abstracts. Aichi, Japan, p. 44.
Kitalong, C., Tadao, V R., Hillmann, A., Balick, M., Kennelly, E., 2012. Phytochemical analysis of Palauan traditional medicinal plant Phaleria nisidai to determine therapeutic dosing of Mangiferin, A C-glucosyl xanthone. Planta Med. 78, PF1.
Kitalong, C., 2014. Ethnomedical, Ecological and Phytochemical Studies of the Palauan Flora (Ph.D thesis). New York Graduate Institute, New York.
Kulakowski, D., Kitalong, C., Negrin, A., Tadao, V. R., Balick, M. J., Kennelly, E. J., 2015. Traditional preparation of Phaleria nisidai, a Palauan tea, reduces exposure to toxic daphnane-type diterpene esters while maintaining immunomodulatory activity. J. Ethnopharmacol. 173, 273-279.
Li, Y., Huang, T. H., Yamahara, J., 2008. Salacia root, a unique Ayurvedic medicine, meets multiple targets in diabetes and obesity. Life Sci. 82, 1045-1049.
Matsuda, H., Tokunaga, M., Iwahashi, H., Naruto, S., Yagi, H., Masuko, T., Kubo, M., 2005. Studies on Palauan medicinal herbs. II. activation of mouse macrophages RAW 264.7 by Ongael, leaves of Phaleria cumingii (Meisn.) F. Vill. and its acylglucosylsterols. Biol. Pharm. Bull. 28, 929-933.
Miura, T., Ichiki, H., Hashimoto, I., Iwamoto, N., Kao, M., Kubo, M., Ishihara, E., Komatsu, Y., Okada, M., Ishida, T., Tanigawa, K., 2001. Antidiabetic Activity of a Xanthone Compound, Mangiferin. Phytomedicine 8 (2), 85-87.
Østoft, S. H., Bagger, J. I., Hansen, T., Pedersen, O., Faber, J., Hoist, J. J., Knop, F. K., Vilsbøll, T., 2014. Glucose-lowering effects and low risk of hypoglycemia in patients with maturity onset diabetes of the young when treated with a GLP-1 receptor agonist: a double-blind, randomized, crossover trial. Diabetes Care. 37, 1797-1805.
American Diabetes Association, 2009. Standards of medical care in diabetes. Diabetes Care 32, S6S12. Stohs, S. J., Ray, S., 2015. Anti-diabetic and anti-hyperlipidemic effects and safety of Salacia reticulata and related species. Phytother. Res. 29, 986-995.
WHO, 2004. Guidelines on Safety Monitoring of Herbal Medicines in Pharmacovigilance Systems. In: W.H. (Ed.), Organization. Geneva, pp. 1-47.

1. A method for preparation of an herbal composition, the method comprising:
adding Phaleria nisidai into water wherein ratio of the Phaleria nisidai to the water is 1 to 10:10 to 2,000 weight/volume (w/v) and boiling the water with the Phaleria nisidai for 1 minute to 3 hours.

2. The method according to claim 1, wherein the ratio is 6:1,000 w/v.

3. The method according to claim 1, wherein the water with the Phaleria nisidai is boiled for 1 minute to 1 hour.

4. A method for preparation of an herbal composition, the method comprising:
adding Phaleria nisidai into water wherein ratio of the Phaleria nisidai to the water is 1 to 10:10 to 2,000 w/v and
steeping Phaleria nisidai in the water for 1 minute to 3 hours.

5. The method according to claim 4, wherein the ratio is 6:1,000 w/v.

6. The method according to claim 4, wherein the water with the Phaleria nisidai is steeped for 1 minute to 1 hour.

7. A method of treatment of metabolic syndromes for a subject, the method comprising:
administrating a first composition comprising hypoglycemic agent as an active ingredient to the subject; and
administrating a second composition comprising Phaleria nisidai as an adjuvant active ingredient before a meal.

8. The method according to claim 7, wherein the subject is administered the compositions before the meal from 1 minute to 3 hours.

9. The method according to claim 7, wherein the subject is administered the compositions before the meal from 1 minute to 2 hours.

10. The method according to claim 7, wherein the subject is administered the compositions before the meal from 1 minute to 1 hour.

11. The method according to claim 7, wherein the subject is administered the compositions 30 minutes before the meal.

12. The method according to claim 7, wherein the hypoglycemic agent is selected from the group consisting of Metformin, Glyburide, Glucovanse, and Micronase.
